SEQUENCE LISTING (38 sequences)

SEQ ID NO: 1 - RNA, 22 nt, Homo sapiens
  source 1..22: mol_type = other RNA
aaacaaacatggtgcacttctt

SEQ ID NO: 2 - RNA, 23 nt, Homo sapiens
  source 1..23: mol_type = other RNA
aatgacacgatcactcccgttga

SEQ ID NO: 3 - RNA, 21 nt, Homo sapiens
  source 1..21: mol_type = other RNA
cattattacttttggtacgcg

SEQ ID NO: 4 - RNA, 22 nt, Homo sapiens
  source 1..22: mol_type = other RNA
ttccctttgtcatcctatgcct

SEQ ID NO: 5 - RNA, 19 nt, Homo sapiens
  source 1..19: mol_type = other RNA
aaaagctgggttgagagga

SEQ ID NO: 6 - RNA, 24 nt, Homo sapiens
  source 1..24: mol_type = other RNA
tggaagactagtgattttgttgtt

SEQ ID NO: 7 - RNA, 22 nt, Homo sapiens
  source 1..22: mol_type = other RNA
ctgaagctcagagggctctgat

SEQ ID NO: 8 - RNA, 22 nt, Homo sapiens
  source 1..22: mol_type = other RNA
taacagtctacagccatggtcg

SEQ ID NO: 9 - RNA, 20 nt, Homo sapiens
  source 1..20: mol_type = other RNA
tgttcctctgtctcccagac

SEQ ID NO: 10 - RNA, 22 nt, Homo sapiens
  source 1..22: mol_type = other RNA
ggaggaaccttggagcttcggc

SEQ ID NO: 11 - RNA, 21 nt, Homo sapiens
  source 1..21: mol_type = other RNA
taaagtgctgacagtgcagat

SEQ ID NO: 12 - RNA, 22 nt, Homo sapiens
  source 1..22: mol_type = other RNA
tgtgacagattgataactgaaa

SEQ ID NO: 13 - RNA, 22 nt, Homo sapiens
  source 1..22: mol_type = other RNA
agatcgaccgtgttatattcgc

SEQ ID NO: 14 - RNA, 22 nt, Homo sapiens
  source 1..22: mol_type = other RNA
tgcctactgagctgaaacacag

SEQ ID NO: 15 - RNA, 22 nt, Homo sapiens
  source 1..22: mol_type = other RNA
tccgtctcagttactttatagc

SEQ ID NO: 16 - RNA, 21 nt, Homo sapiens
  source 1..21: mol_type = other RNA
atgacctatgaattgacagac

SEQ ID NO: 17 - RNA, 23 nt, Homo sapiens
  source 1..23: mol_type = other RNA
caggcagtgactgttcagacgtc

SEQ ID NO: 18 - RNA, 24 nt, Homo sapiens
  source 1..24: mol_type = other RNA
tgaagcgcctgtgctctgccgaga

SEQ ID NO: 19 - RNA, 21 nt, Homo sapiens
  source 1..21: mol_type = other RNA
aattacagattgtctcagaga

SEQ ID NO: 20 - RNA, 23 nt, Homo sapiens
  source 1..23: mol_type = other RNA
agcagcattgtacagggctatca

SEQ ID NO: 21 - RNA, 21 nt, Homo sapiens
  source 1..21: mol_type = other RNA
cttgggagccctgttagactc

SEQ ID NO: 22 - RNA, 22 nt, Homo sapiens
  source 1..22: mol_type = other RNA
ttaattttttgtttcggtcact

SEQ ID NO: 23 - RNA, 22 nt, Homo sapiens
  source 1..22: mol_type = other RNA
ctatacagtctactgtctttcc

SEQ ID NO: 24 - RNA, 22 nt, Homo sapiens
  source 1..22: mol_type = other RNA
ctatacaatctattgccttccc

SEQ ID NO: 25 - RNA, 22 nt, Homo sapiens
  source 1..22: mol_type = other RNA
ttcaacgggtatttattgagca

SEQ ID NO: 26 - RNA, 23 nt, Homo sapiens
  source 1..23: mol_type = other RNA
tgtgcaaatctatgcaaaactga

SEQ ID NO: 27 - RNA, 23 nt, Homo sapiens
  source 1..23: mol_type = other RNA
ctggagatatggaagagctgtgt

SEQ ID NO: 28 - RNA, 21 nt, Homo sapiens
  source 1..21: mol_type = other RNA
tcgaccggacctcgaccggct

SEQ ID NO: 29 - RNA, 19 nt, Homo sapiens
  source 1..19: mol_type = other RNA
atcccaccactgccaccat

SEQ ID NO: 30 - RNA, 22 nt, Homo sapiens
  source 1..22: mol_type = other RNA
tggagtgtgacaatggtgtttg

SEQ ID NO: 31 - RNA, 22 nt, Homo sapiens
  source 1..22: mol_type = other RNA
gccctgtggactcagttctggt

SEQ ID NO: 32 - RNA, 21 nt, Homo sapiens
  source 1..21: mol_type = other RNA
cataaagtagaaagcactact

SEQ ID NO: 33 - RNA, 22 nt, Homo sapiens
  source 1..22: mol_type = other RNA
actggacttggagtcagaaggc

SEQ ID NO: 34 - RNA, 21 nt, Homo sapiens
  source 1..21: mol_type = other RNA
tgagatgaagcactgtagctc

SEQ ID NO: 35 - RNA, 22 nt, Homo sapiens
  source 1..22: mol_type = other RNA
ggtgcagtgctgcatctctggt

SEQ ID NO: 36 - RNA, 22 nt, Homo sapiens
  source 1..22: mol_type = other RNA
aatcatacacggttgacctatt

SEQ ID NO: 37 - RNA, 22 nt, Homo sapiens
  source 1..22: mol_type = other RNA
gagggttgggtggaggctctcc

SEQ ID NO: 38 - RNA, 22 nt, Homo sapiens
  source 1..22: mol_type = other RNA
ggattcctggaaatactgttct

(19) United States
(12) Patent Application Publication
(10) Pub. No.: US 2024/0200072 A1
(43) Pub. Date: Jun. 20, 2024

(54) Allogeneic Cartilage Regeneration

(71) Applicant: Tarachon, LLC, Brooklyn, NY (US)

(72) Inventor: Tara SHARIF, Brooklyn, NY (US)

(73) Assignee: Tarachon, LLC, Brooklyn, NY (US)

(21) Appl. No.: 18/536,346

(22) Filed: Dec. 12, 2023

Related U.S. Application Data
(60) Provisional application No. 63/386,926, filed on Dec. 12, 2022.

(51) Int. Cl. C12N 15/113; A61K 9/50; A61K 45/06; C12Q 1/6874; C12Q 1/6883

(52) U.S. Cl. CPC C12N 15/113; A61K 9/5068; A61K 45/06; C12Q 1/6874; C12Q 1/6883; C12N 2310/141; C12N 2310/313; C12N 2310/315; C12Q 2600/178

(57) ABSTRACT

Pharmaceutical compositions and methods for treating and/or preventing arthritis, including osteoarthritis and/or rheumatoid arthritis in a patient.

This application claims benefit of U.S. Ser. No. 63/386,926 filed Dec. 12, 2022, the entirety of which is incorporated herein by reference.

### INCORPORATION BY REFERENCE OF SEQUENCE LISTING PROVIDED AS A SEQUENCE LISTING XML FILE

The Sequence Listing XML associated with this application is provided electronically in XML file format and is hereby incorporated by reference into the specification. The name of the XML file containing the Sequence Listing XML is 42310011PV01SEQ_Listing created on Dec. 21, 2023 and having a size of 34 KB. The contents of the Sequence Listing XML are incorporated herein by reference in their entirety.

### SPECIFICATION

The present disclosure relates generally to making a pharmaceutical composition of synthetic biologics for cartilage repair, and to methods of allogeneic treatment of a subject in need of cartilage repair, by administering an effective dose of a formulation either by direct injection or by the use of, for example, scaffolds or glues or the like.

### BACKGROUND

Osteoarthritis (OA) is the most common form of arthritis. It develops when the protective hyaline cartilage on the ends of the bones wear down in time. Repetitive movements, heavy lifting, weakness of muscles associated with the joints and athletic injuries can also lead to cartilage breakdown and OA. Osteoarthritis causes pain, inflammation, and reduced motion in all joints, but mostly in the joints of the knees, hips, shoulders, hands and spine.

An estimated 10%-15% of all adults aged over 60 will develop some degree of OA (over 30 million adults in the US), with prevalence being higher among women, and on the rise due to the ageing of the populations and obesity. OA is also a source of morbidity and economic loss in the race horse populations.

Aside from osteoarthritis, a large population of younger subjects are afflicted with injuries to cartilage or ligaments of the joints. Common acute injuries of the joint involve damage to anterior and posterior cruciate ligament (ACL and PCL), medial and lateral collateral ligaments (MCL and LCL), and menisci.

Although pain, reduced mobility and other symptoms of damaged joints can be temporarily managed by routine modalities (pain killers, injection of steroids, hyaluronic acid in the joints, etc.), the underlying cause of the disorder including articular cartilage damage persists.

Advanced cell therapy treatments have sought to regenerate the damaged cartilage and other components of the joints and restore the normal joint functions. Cells commonly used in regeneration include autologous mesenchyme stem cells derived from bone marrow, adipose tissue and full-grown cartilage. Issues associated with the use of these sources include limited number of relevant stem cells, and invasive collection practices.

Heterologous sources are also used for stem cell therapy. These sources include umbilical, embryonic or placental tissues. These modalities involve complicated harvesting processes, possible immunological reactions, and sub-optimal number of compatible cells.

Autologous Synovial Fluid—Derived Mesenchyme Stem Cells (SF-MSC) have shown greatest potential for repair of the damaged cartilage tissues. Chondrogenic Mesenchyme Stem Cells are higher in number in synovial fluid than other sources and can be harvested non-invasively from the synovial cavity during an office visit. However, the direct use of autologous Mesenchyme Stem Cells has limitations, including lack of scalability, patient morbidity, lengthy processing time and high cost.

Thus, there exists a need for methods of biological treatment that can be used allogeneically, is more effective and can use the advantages of stem cell-treatment without bearing the lengthy processing time and costs associated with the direct use of these cells, while diminishing immunoreactive side effects of whole cell treatment in the patients.

The present disclosure provides compositions and methods for cartilage regeneration by the use of, for example, a selected subset of synovial fluid mesenchyme stem cell—derived exosomes and/or micro RNAs derived from these exosomes.

All references cited herein are incorporated herein by reference in their entireties.

### BRIEF SUMMARY

Synovial Fluid Mesenchymal Stem Cells (SF-MSCs) have shown great potential for cartilage regeneration and repair. However, the direct use of these cells is associated with lack of scalability and cost and morbidity limitations. Recent evidence has shown that the activation and recruitment of stem cells required for tissue regeneration after an injury, are mediated and orchestrated by exosomes.

Exosomes are membrane-bound 30-150 nm wide extracellular vesicles (EVs) that are produced by multivesicular bodies (MVB) prior to extracellular secretin. Exosomes are found in biological fluids including blood, urine and cerebrospinal fluid. They are also released in media of cultured cells.

Exosomes derived from SF-MSCs display similar biological functions to the cells they were originated from and carry signaling factors that play important roles in intercellular communication and orchestration of repair and response to injury. Exosomes have no reported immunologic or tumor induction adverse effects.

Despite an increasing number of studies pointing to the therapeutic effects of exosomes, the underlying mechanism of action of exosomes remains unclear. SF-MSC exosomes carry peptides, miRNA, lncRNA, tRNA, and lipid molecules which can initiate a repair cascade by promoting migration, proliferation, and differentiation of adjacent stem cells. This synchronized set of events could lead to new cartilage formation and regeneration.

MicroRNAs (miRNAs), are single-stranded non-coding RNAs of about 20-24 nucleotides in length that bind to the 3′-UTR of their target mRNAs and regulate translation. Over 90% of human KEGG pathways either contain genes which are targeted by miRNAs or harbor these molecules. In a broad array of processes, miRNAs can fine tune or restrict cellular properties by targeting important transcription factors or key pathways. Several studies have demonstrated the importance of miRNAs for tissue development, differentiation, and repair.

The cartilage repair mechanism attributed to SF-MSCs and their exosomes seem to take place at a faster pace in the young, yet progressively decline in age. See for example, U.S. 2021/0268031, incorporated by reference herein in its entirety. In our prior work we showed statistically significant declining number of SF-MSC harvested from the knee joints of patients from different age groups (See for example U.S. 2021/0268031 at FIG. 8).

The variance in the number and types of exosomes and their miRNA phenotype profiles were analyzed to determine the implication of age in the ability of SF-MSCs and their exosomes to repair cartilage damage. The number of exosomes secreted from a normalized number of SF-MSCs is higher in case of the cells derived from younger vs older patients. In addition, there is higher labelling efficiency (higher percentage of intact exosomes) of the exosomes derived from “young” vs “old” SF-MSCs. (See FIG. 1).

For microRNA analysis, exosomal RNAs sourced from 6 subjects of varying ages were isolated using Isolation Kit (NorgenBiotek) and analyzed using Next Generation Sequencing (MGS), NextSeq 500/550 High Output Kit v2 (75-Cycle Kit). Library preparation workflow included 3′ and 5′ adapter ligation, followed by reverse transcription and the indexing PCR. Library QC was performed using Agilent Bioanalyzer pico chip to estimate library size and concentration. Libraries were then pooled, denatured and diluted to required concentration. Libraries were applied onto a flowcell and sequenced using Illumina platform. The reads were then mapped to the whole genome. Large RNAs (more than 100 nucleotides) were filtered out and 40-50 nucleotide RNAs (miRNA, some tRNA, etc.) were mapped to different biotypes. Read counts for small RNAs were normalized using Counts Per Million as normalization method where the read of a specific small RNA species is divided by the total number of mapped reads and multiplied by one million. (See FIG. 10).

Read counts mapped to different biotypes based on normalized raw indicated that the most frequent occurrence of RNA for all groups was at 22 nucleotides (miRNA) peak. Second highest peak occurred at 32 nucleotides (tRNA). Both peaks showed higher number of read in group B (young) than group A (old), indicating higher miRNA and tRNA counts/million in Group B. (See FIG. 2).

For differential Expression analysis trimmed mean of M-values (TMM) normalization method was used to overcome under-sampling effects and reduce false-positive rate. Using the TMM method, the variabilities among the miRNAs of group “young”, B vs group “old”, A were assessed. The study demonstrated that miRNAs with lowest count showed higher variability of expression among groups than those that occur at higher count (See FIG. 3)

A Volcano plot was produced by plotting −log 10 of the false discovery rate (FDR, y-axis) and the log 2 fold change between the groups A and B (Log 2 FC (A/B). Markers on the left represented higher variability of miRNA expression in Group B than A (A/B<1). (See FIG. 4)

Venn Diagram, showed the logical relationships between min 5 TMM normalized counts miRNAs of all groups. Of the total miRNAs, 270 were commonly expressed in both groups A, “old” and B, “young”. 41 miRNAs were unique to group B, “young” and 19 miRNAs were unique to group-A, “old”. (See FIG. 5)

Bioinformatics analysis was performed to rank the miRNAs unique to groups A, “old” and group B, “young” in order of highest to lowest concentration. (See FIGS. 6 and 7 respectively).

The nucleotide sequence of the miRNAs unique to group B was determined “young” by reference to the miRBase: the microRNA database (www.mirbase.org). (See FIG. 8). The nucleotide sequence of exemplary miRNAs that are overrepresented in group B (young) vs Group A (old) was also determined. (See FIG. 11). In certain embodiments, the miRNAs considered “unique” or “overrepresented” in the young group had a higher concentration than in the old group. In certain embodiments, the miRNAs considered “unique” or “overrepresented” in the young group had about a 1% higher concentration than in the old group; In certain embodiments, the miRNAs considered “unique” or “overrepresented” in the young group had about a 2% higher concentration than in the old group; In certain embodiments, the miRNAs considered “unique” or “overrepresented” in the young group had about a 5% higher concentration than in the old group; In certain embodiments, the miRNAs considered “unique” or “overrepresented” in the young group had about a 10% higher concentration than in the old group; In certain embodiments, the miRNAs considered “unique” or “overrepresented” in the young group had about a 20% higher concentration than in the old group; In certain embodiments, the miRNAs considered “unique” or “overrepresented” in the young group had about a 30% higher concentration than in the old group; In certain embodiments, the miRNAs considered “unique” or “overrepresented” in the young group had about a 40% higher concentration than in the old group. In certain embodiments, the miRNAs considered “unique” or “overrepresented” in the young group had a higher concentration than in the old group by about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, about 99.8% or about 100%. In certain embodiments, the miRNAs considered “unique” or “overrepresented” in the young group had a fold increase in concentration of about 0.01 fold, about 0.02 fold, about 0.05 fold, about 0.1 fold, about 0.2 fold, about 0.3 fold, about 0.4 fold, about 0.5 fold, about 0.6 fold, about 0.7 fold, about 0.8 fold, about 0.9 fold, about 1 fold, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, or more, compared to the old group.

Accordingly, the disclosure provides for methods of synthesizing a pharmaceutical formulation of miRNAs, and/or exosomes comprising miRNAs, inferred from those unique or overrepresented to the “young” group, and method of allogeneic cartilage repair by administering the synthesized formulation to a subject in need of cartilage repair either by direct injection or by the use of scaffolds or glues or the like.

The compositions and methods as disclosed herein are for the prevention and/or treatment of arthritis, such as osteoarthritis, and without being bound by any theory, can elicit an effective cartilage repair response through activation and mobilization of endogenous stem/progenitor cells, without the need to transplant cells or exosomes, to save cost and morbidity and to expand the utility of such treatments.”

The disclosure provides a method of treating, preventing, and/or controlling arthritis and the pain associated with arthritis in a patient, the method comprising: selecting a patient in need of treating, preventing, and/or controlling arthritis, torn or damaged meniscus cartilage, torn or damaged labrum, subchondral bone edema, torn or damaged joint ligament, torn or damaged patellar cartilage, or an external injury to the joint; administering to the patient a therapeutically effective amount of a pharmaceutical composition comprising at least one of the following: (i) at least one miRNA selected from the group consisting of SEQ ID NO: 1 to SEQ ID NO: 38, and combinations thereof; (ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect; (iii) at least one miRNA having 80% or more sequence identity to a miRNA selected from the group consisting of SEQ ID NO: 1 to SEQ ID NO: 38, and combinations thereof, and having a therapeutic effect; (iv) a nucleic acid encoding at least one miRNA selected from the group consisting of SEQ ID NO: 1 to SEQ ID NO: 38, and combinations thereof; (v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect; (vi) a nucleic acid encoding at least one miRNA having 80% or more sequence identity to a miRNA selected from the group consisting of SEQ ID NO: 1 to SEQ ID NO: 38, and combinations thereof, and having a therapeutic effect, or (vii) combinations thereof. The disclosure provides a method wherein said arthritis is osteoarthritis or rheumatoid arthritis. The disclosure provides a method wherein the subject in need of treatment has low chondrogenic potential or has small stem cell population as in the gender-specific and age-specific stem cell count variabilities. The disclosure provides a method wherein the subject is in need of tissue reconstruction or tissue augmentation therapy, including the treatment of acute and chronic wounds that have afflicted layers of connective and epidermal tissue of the body, or for aesthetic reasons, including reduction of wrinkles, grooves, scars, acne scars, traumatic scars, sequelae of cellulite, as well as for other irregularities of the skin, to give a smoother skin. The disclosure provides a method wherein the patient in need of treating, preventing, and/or controlling arthritis and the pain associated with arthritis is selected from the group consisting of a human, a horse, or a companion animal. The disclosure provides a method wherein said miRNA is chemically modified. The disclosure provides a method wherein said chemical modification is one or more chemical modifications selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof. The disclosure provides a method wherein the miRNA comprises a modified backbone. The disclosure provides a method wherein the miRNA comprises a phosphorothioate backbone or a phosphorodithioate backbone. The disclosure provides a method wherein the pharmaceutical composition further comprises a nucleic acid transfection agent. The disclosure provides a method wherein said transfection agent is a lipid-based transfection agent, a polymer-based transfection agent, a magnetic particle-based transfection agent, an exosome for nucleic acid delivery, or a viral protein for nucleic acid delivery. The disclosure provides a method wherein the pharmaceutical composition is for topical administration. The disclosure provides a method wherein the pharmaceutical composition is formulated for parenteral administration. The disclosure provides a method wherein the pharmaceutical composition is formulated for intra-articular, intravenous, intramuscular, sub-cutaneous, or intraperitoneal injection. The disclosure provides a method wherein said pharmaceutical composition is administered by a mode selected from the group consisting of injection, a scaffold, a 3-D scaffold, a matrix and a glue, carboplasty, and combinations thereof. The disclosure provides a method further comprising co-administration with other antiarthritis agents. The disclosure provides a method wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, and combinations thereof.

The disclosure provides a pharmaceutical composition for treating, preventing, and/or controlling arthritis in a patient comprising at least one of the following: (i) at least one miRNA selected from the group consisting of SEQ ID NO: 1 to SEQ ID NO: 38, and combinations thereof; (ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect; (iii) at least one miRNA having 80% or more sequence identity to a miRNA selected from the group consisting of SEQ ID NO: 1 to SEQ ID NO: 38, and combinations thereof, and having a therapeutic effect; (iv) a nucleic acid encoding at least one miRNA selected from the group consisting of SEQ ID NO: 1 to SEQ ID NO: 38, and combinations thereof; (v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect; (vi) a nucleic acid encoding at least one miRNA having 80% or more sequence identity to a miRNA selected from the group consisting of SEQ ID NO: 1 to SEQ ID NO: 38, and combinations thereof, and having a therapeutic effect, or (vii) combinations thereof. The disclosure provides a pharmaceutical composition wherein said arthritis is osteoarthritis or rheumatoid arthritis. The disclosure provides a pharmaceutical composition wherein said miRNA is a pri-miRNA, a pre-miRNA, a double-stranded miRNA, a single-strand miRNA expressed from the 5′-end of a pre-miRNA, or a single-strand miRNA expressed from the 3′-end of a pre-miRNA. The disclosure provides a pharmaceutical composition wherein said miRNA is chemically modified. The disclosure provides a pharmaceutical composition wherein said chemical modification is selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof. The disclosure provides a pharmaceutical composition wherein the miRNA comprises a modified backbone. The disclosure provides a pharmaceutical composition wherein the miRNA comprises a phosphorothioate backbone or a phosphorodithioate backbone. The disclosure provides a pharmaceutical composition wherein the pharmaceutical composition further comprises a nucleic acid transfection agent. The disclosure provides a pharmaceutical composition wherein said transfection agent is a lipid-based transfection agent, a polymer-based transfection agent, a magnetic particle-based transfection agent, an exosome for nucleic acid delivery, or a viral protein for nucleic acid delivery. The disclosure provides a pharmaceutical composition wherein the composition is for topical administration. The disclosure provides a pharmaceutical composition wherein the composition is formulated for parenteral administration. The disclosure provides a pharmaceutical composition wherein the composition is formulated for intra-articular, intravenous, intramuscular, sub-cutaneous, or intraperitoneal injection. The disclosure provides a pharmaceutical composition wherein the composition is co-administered in combination with other antiarthritis agents. The disclosure provides a pharmaceutical composition wherein said other antiarthritis agents are one or more antiarthritis agents selected from the group consisting of wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof.

The disclosure provides a pharmaceutical composition comprising an exosome and an excipient, wherein the exosome comprises: (i) at least one miRNA selected from the group consisting of SEQ ID NO: 1 to SEQ ID NO: 38, and combinations thereof; (ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect; (iii) at least one miRNA having 80% or more sequence identity to a miRNA selected from the group consisting of SEQ ID NO: 1 to SEQ ID NO: 38, and combinations thereof, and having a therapeutic effect; (iv) a nucleic acid encoding at least one miRNA selected from the group consisting of SEQ ID NO: 1 to SEQ ID NO: 38, and combinations thereof; (v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect; or (vi) a nucleic acid encoding at least one miRNA having 80% or more sequence identity to a miRNA selected from the group consisting of SEQ ID NO: 1 to SEQ ID NO: 38, and combinations thereof, and having a therapeutic effect. The disclosure provides a pharmaceutical composition wherein the composition is formulated for parenteral administration. The disclosure provides a pharmaceutical composition wherein the composition is formulated for intra-articular, intravenous, intramuscular, sub-cutaneous, or intraperitoneal injection. The disclosure provides a pharmaceutical composition wherein the miRNA comprises a modified backbone. The disclosure provides a pharmaceutical composition wherein the miRNA comprises a phosphorothioate backbone or a phosphorodithioate backbone. The disclosure provides a pharmaceutical composition wherein the miRNA comprises one or more chemical modifications selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof. The disclosure provides a pharmaceutical composition wherein the composition is used in combination with other antiarthritis agents. The disclosure provides a pharmaceutical composition wherein said other antiarthritis agents are one or more antiarthritis agents selected from the group consisting of wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof. The disclosure provides a pharmaceutical composition wherein the pharmaceutical composition is in a form selected from the group consisting of a sterile aqueous solution, a sterile dispersion, a sterile powder, a lyophilized form, a gel, a paste, a wax, a cream, a spray, a liquid, a foam, a lotion, an ointment, an injectable solution, an injectable dispersion, a topical solution, a transdermal form, a transdermal patch, a powder, a vapor, a tincture, and combinations thereof.

The disclosure provides a method of identification of signaling factors/biologic factors which can be used to induce cartilage growth in a patient in need thereof, the method comprising: Providing an older patient; isolating synovial fluid from the older patient; Isolating signaling factors from the synovial fluid, or cells or cell derivatives of synovial fluid, adipose tissue or bone marrow of adults, cells from embryo, fetuses, placentas, umbilical cord, Wharton Jelly or a mixture thereof, of the older patient; providing a younger patient; isolating synovial fluid from the younger patient' isolating signaling factors from the synovial fluid, or cells or cell derivatives of synovial fluid, adipose tissue or bone marrow of adults, cells from embryo, fetuses, placentas, umbilical cord, Wharton Jelly or a mixture thereof, of the younger patient; performing a comparison of the signaling factors isolated from the synovial fluid, or cells or cell derivatives of synovial fluid, adipose tissue or bone marrow of adults, cells from embryo, fetuses, placentas, umbilical cord, Wharton Jelly or a mixture thereof of the younger patient to the signaling factors isolated from the synovial fluid, or cells or cell derivatives of synovial fluid, adipose tissue or bone marrow of adults, cells from embryo, fetuses, placentas, umbilical cord, Wharton Jelly or a mixture thereof from the older patient; identifying the signaling factors which are unique to the synovial fluid, or cells or cell derivatives of synovial fluid, adipose tissue or bone marrow of adults, cells from embryo, fetuses, placentas, umbilical cord, Wharton Jelly or a mixture thereof of the younger patient; wherein the unique signaling factors from the younger patient are useful for growing cartilage. The disclosure provides a method wherein the younger patient is under about 25 years of age. The disclosure provides a method wherein the older patient is over about 55 years of age. The disclosure provides a method wherein the signaling factors are selected from the group consisting of Synovial Fluid-Derived Mesenchyme Stem Cells, Exosomes, Exosomal RNA, Exosomal miRNA, Exosomal tRNA, Exosomal peptides and combinations thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### BRIEF DESCRIPTION OF SEVERAL VIEWS OF THE DRAWINGS

The invention will be described in conjunction with the following drawings in which like reference numerals designate like elements and wherein:

FIG. 1 is a Table showing the Exosome Isolation information for each subject.

FIG. 2 is a chart showing the read length distribution of RNA biotypes based on normalized raw reads.

FIG. 3 is a chart showing the correlation between miRNA counts of groups A and B using the TMM (trimmed mean of M-values) normalization method.

FIG. 4 is a chart showing a Volcano plot of Differential Expression (DE) of normalized miRNAs of groups A and B. Volcano plot was produced by plotting −log 10 of the false discovery rate (FDR, y-axis) and the log 2 fold change between the compared groups (log 2foldchange, x-axis).

FIG. 5 is a Venn Diagram showing the number of unique sequences, TMM>5.

FIG. 6 is a chart showing Group A unique miRNAs, TMM>5.

FIG. 7 is a chart showing Group B unique miRNAs, TMM>5.

FIG. 8 is a Table Showing the nucleotide sequences of miRNAs from younger patients.

FIG. 9 is a chart showing Differentially Expressed tRNAs with CPM>5—Groups A and B.

FIG. 10 is a chart showing Exosome NGS RNA Run and Mapping Summary.

FIG. 11 is a chart showing exemplary miRNAs that are overrepresented in group B (young) vs Group A (old).

### DETAILED DESCRIPTION

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, sheep, or cow) or a pet (e.g., a dog or cat). The non-human mammal may be a domestic pet, or animal kept for commercial purposes, e.g., a racehorse, or farming livestock or animals such as pigs, sheep or cattle. As such the disclosure may have veterinary applications. Non-human mammals include rabbits, guinea pigs, rats, mice or other rodents (including any animal in the order Rodentia), cats, dogs, pigs, sheep, goats, cattle (including cows or any animal in the order Bos), horse (including any animal in the order Equidae), donkey, and non-human primates. The subject may be male or female. In a specific embodiment, the subject is an elderly human. In another embodiment, the subject is a human adult. In another embodiment, the subject is a human child. In yet another embodiment, the subject is a human infant. As used herein the term “younger patient” refers to patient under about 25 years of age. As used herein the term “older patient” refers to a patient over about 55 years of age.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein the term “combination administration” of a compound, therapeutic agent or known drug with the combination of the present invention means administration of the drug and the one or more compounds at such time that both the known drug and/or combination will have a therapeutic effect. In some cases, this therapeutic effect will be synergistic. Such concomitant administration can involve concurrent (i.e., at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

As used herein, the term “multi-particulates” refers to one or more unit dosage systems such as, but not limited to, pellets, beads, spheres, mini-tablets, seeds, spheroids or granules with modified drug release profile. The multi-particulates comprise a drug-release controlling and/or drug-protecting film or matrix, such as a polymeric film or matrix, whose intactness or efficiency is susceptible to certain conditions such as heat or mechanical forces that may occur during post-processing. The expression “core material” describes the nature of the interior part of multi-particulates that may also comprise a functional coat. Exemplary “core-materials” may be pellets (spherical matrix systems that contain a drug and further excipients), granules (less spherical particles that are almost entirely composed of drug) or nonpareils (spherical particles without drug).

As used herein, the term “about” when used in conjunction with a stated numerical value or range has the meaning reasonably ascribed to it by a person skilled in the art, i.e., denoting somewhat more or somewhat less than the stated value or range.

### Indications

The present disclosure provides compositions and methods for cartilage regeneration by the use of, for example, a selected subset of synovial fluid mesenchyme stem cell—derived exosomes and/or micro RNAs derived from these exosomes for the prevention and/or treatment of arthritis and arthritic conditions, such as osteoarthritis and rheumatoid arthritis. Osteoarthritis (OA), which is also called “degenerative joint disease” or arthrosis, is one of the most common disorders of the musculo-skeletal system. The most common joints affected by OA are the knees, hands, hips and big toes. The exact causes of this condition are unknown and difficult to resolve as multiple factors play a role in the initiation and progression of the disease. It is associated with a certain genetic background as individuals who have a history of OA in their family have increased risk of developing the disease, but also many other factors play a role as exemplified by the susceptibility of people who have previously had a serious injury, such as ligament or meniscal damage, or certain forms of joint-surgery to develop OA.

Rheumatoid arthritis (RA) is an inflammatory condition where articular cartilage of affected joints is being degraded by an active process involving cells of the immune system as well as the tissues of the joint (i.e. the synovial membrane, the cartilage and subchondral bone). The etiology of RA is complex and a number of environmental and genetic factors have been suggested a role in the development of the disease.

In certain embodiments as disclosed herein, the condition to be prevented and/or treated is selected from the group consisting of: osteoarthritic pain, rheumatoid arthritis pain, juvenile chronic arthritis associated pain, juvenile idiopathic arthritis associated pain, Spondyloarthropathies (such as ankylosing spondylitis (Mb Bechterew) and reactive arthritis (Reiter's syndrome)) associated pain, pain associated with psoriatic arthritis, gout pain, pain associated with pseudogout (pyrophosphate arthritis), pain associated with systemic lupus erythematosus (SLE), pain associated with systemic sclerosis (scleroderma), pain associated with Behçet's disease, pain associated with relapsing polychondritis, pain associated with adult Still's disease, pain associated with transient regional osteoporosis, pain associated with neuropathic arthropathy, pain associated with sarcoidosis, arthritic pain, rheumatic pain, joint pain, osteoarthritic joint pain, rheumatoid arthritic joint pain, juvenile chronic arthritis associated joint pain, juvenile idiopathic arthritis associated joint pain, Spondyloarthropathies (such as ankylosing spondylitis (Mb Bechterew) and reactive arthritis (Reiter's syndrome)) associated joint pain, joint pain associated with psoriatic arthritis, gout joint pain, joint pain associated with pseudogout (pyrophosphate arthritis), joint pain associated with systemic lupus erythematosus (SLE), joint pain associated with systemic sclerosis (scleroderma), joint pain associated with Behçet's disease, joint pain associated with relapsing polychondritis, joint pain associated with adult Still's disease, joint pain associated with transient regional osteoporosis, joint pain associated with neuropathic arthropathy, joint pain associated with sarcoidosis, arthritic joint pain, rheumatic joint pain, acute pain, acute joint pain, chronic pain, chronic joint pain, inflammatory pain, inflammatory joint pain, mechanical pain, mechanical joint pain, pain associated with the fibromyalgia syndrome (FMS), pain associated with polymyalgia rheumatica, monarticular joint pain, polyarticular joint pain, nociceptiv pain, neuropathic pain, psychogenous pain, pain of unknown etiology.

In certain embodiments, the disclosure provides for skin rejuvenation and for cosmetic reasons, which includes the correction of contour deformities, reduction of wrinkles, acne scars, pits, surgically induced irregularities and other skin defects. An aging dermis loses collagen and elastin over time, as a result of which the skin becomes thinner and irregular resulting in wrinkles. Wrinkles can be only fine expression lines or deep wrinkles on the skin. Other factors that favor wrinkles are smoking, sun damage (photoaging), dryness and loss of moisture, skin color (light-colored skin is more likely to develop wrinkles), and inheritance, etc.

The present disclosure provides compositions and methods for use in the administration of at least one miRNA, or nucleic acid encoding one or more miRNAs, or exosome comprising such nucleic acids made by the disclosed methods or pharmaceutical compositions thereof to promote tissue regeneration or augmentation, while at the same time minimizing the need for healing, and the risk of infection. Among several areas of application, it is the treatment of acute and chronic wounds that have afflicted layers of connective and epidermal tissue of the body. Another area of application is as cosmetic treatments, including, for example: reduction of wrinkles, grooves, scars, acne scars, traumatic scars, sequelae of cellulite, as well as for other irregularities of the skin, to give a smoother skin. The disclosure provides, for example, of a collagen-based matrix comprising at least one miRNA, or nucleic acid encoding one or more miRNAs, or exosome comprising such nucleic acids made by the disclosed methods or pharmaceutical compositions thereof. In one embodiment of this disclosure, at least one miRNA, or nucleic acid encoding one or more miRNAs, or exosome comprising such nucleic acids made by the disclosed methods or pharmaceutical compositions thereof is first injected at the site followed by collagen-based matrix implantation. In another embodiment, the at least one miRNA, or nucleic acid encoding one or more miRNAs, or exosome comprising such nucleic acids made by the disclosed methods or pharmaceutical compositions thereof is incorporated into collagen-based matrix. The matrix can also carry important growth factors and cytokines, which promote fibroblast cell migration and proliferation, to the wound area. Microbial infection at the wound site can also be controlled by antibacterial agents.

### Nucleic Acids

Embodiments described herein include methods of treating arthritis such as osteoarthritis or rheumatoid arthritis, comprising administering to a subject an oligonucleotide which may be an miRNA or encodes an miRNA as disclosed herein.

MicroRNAs (miRNAs) are small RNAs of 17-25 nucleotides, which function as regulators of gene expression in eukaryotes. miRNAs are initially expressed in the nucleus as part of long primary transcripts called primary miRNAs (pri-miRNAs). Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (generally nucleotides 2 to 8) which appears to be the primary determinant of the pairing specificity of the miRNA to the 3′ untranslated region (3′-UTR) of a target mRNA.

The chemical structure of the nucleotides of a miRNA molecule or mimics or sources thereof, or of a sense strand or an antisense strand in a mimic of a miRNA or of an isomiRNA, may be modified to increase stability, binding affinity and/or specificity. Said sense strand or antisense strand may comprise or consists of an RNA molecule or preferably a modified RNA molecule. A preferred modified RNA molecule comprises a modified sugar. One example of such modification is the introduction of a 2′-O-methyl or 2′-O-methoxyethyl group or 2′ fluoride group on the nucleic acid to improve nuclease resistance and binding affinity to RNA. Another example of such modification is the introduction of a methylene bridge connecting the 2′-0 atom and the 4′-C atom of the nucleic acid to lock the conformation (Locked Nucleic Acid (LNA)) to improve affinity towards complementary single-stranded RNA. A third example is the introduction of a phosphorothioate group as linker between nucleic acid in the RNA-strand to improve stability against a nuclease attack. A fourth modification is conjugation of a lipophilic moiety on the 3′ end of the molecule, such as cholesterol to improve stability and cellular delivery.

In additional embodiments, an oligonucleotide molecule described herein is modified to increase its stability. In some embodiment, the oligonucleotide molecule is RNA (e.g., miRNA). In some instances, the oligonucleotide molecule is modified by one or more of the modifications described above to increase its stability. In some cases, the oligonucleotide molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the oligonucleotide molecule is modified by 2′-O-methyl and/or 2′-O-methoxyethyl ribose. In some cases, the oligonucleotide molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) oligonucleotide molecule. In some instances, the chirally pure (or stereo pure) oligonucleotide molecule is modified to increase its stability. Suitable modifications to the RNA to increase stability for delivery will be apparent to the skilled person.

The nucleic acid compound may, for example, selected from chemically modified or unmodified DNA, single stranded or double stranded DNA, coding or non-coding DNA, optionally selected from plasmid, (short) oligodeoxynucleotide (i.e. a (short) DNA oligonucleotide), genomic DNA, DNA primers, DNA probes, immunostimulatory DNA, aptamer, or any combination thereof. Alternatively, or in addition, such a nucleic acid molecule may be selected e.g. from any PNA (peptide nucleic acid). Further alternatively, or in addition, and also according to a particularly preferred embodiment, the nucleic acid is selected from chemically modified or unmodified RNA, single-stranded or double-stranded RNA, coding or non-coding RNA, optionally selected from messenger RNA (mRNA), (short) oligoribonucleotide (i.e. a (short) RNA oligonucleotide), viral RNA, replicon RNA, transfer RNA (tRNA), ribosomal RNA (rRNA), immunostimulatory RNA (isRNA), microRNA, small interfering RNA (siRNA), small nuclear RNA (snRNA), small-hairpin RNA (shRNA), or a riboswitch, an RNA aptamer, an RNA decoy, an antisense RNA, a ribozyme, or any combination thereof. Preferably, the nucleic acid molecule of the complex is an RNA. More preferably, the nucleic acid molecule of the complex is a (linear) single-stranded RNA, even more preferably an mRNA or an immunostimulatory RNA.

The present disclosure also provides an expression cassette comprising a sequence encoding a nucleic acid molecule with at least one miRNA, or nucleic acid encoding one or more miRNAs. In certain embodiments, the expression cassette further contains a promoter. In certain embodiments, the promoter is a regulatable promoter. In certain embodiments, the promoter is a constitutive promoter. In certain embodiments, the promoter is a CMV, RSV, or polIII promoter. In certain embodiments, the promoter is not a polIII promoter. The present disclosure provides a vector containing the expression cassette described above. In certain embodiments, the vector is a viral vector. In certain embodiments, the viral vector is an adenoviral, lentiviral, adeno-associated viral (AAV), poliovirus, HSV, or murine Maloney-based viral vector.

In one embodiment, the selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear of nucleolar RNAs or any kind of RNA transcribed by any class of any RNA polymerase I, II or III.

A cell has been “transformed”, “transduced” or “transfected” by an exogenous or heterologous nucleic acid or vector when such nucleic acid has been introduced inside the cell, for example, as a complex with transfection reagents or packaged in viral particles.

As stated, the coding regions of the multiple-promoter miRNA expression cassette are operatively linked to terminator elements. In one embodiment, the terminators comprise stretches of four or more thymidine residues. In another preferred embodiment, the terminator elements used are all different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the miRNA expression cassettes may be configured where multiple cloning sites and/or unique restriction sites are located strategically, such that promoter, miRNA and terminator elements are easily removed or replaced. Moreover, the multiple-promoter miRNA expression cassettes may be assembled from smaller oligonucleotide components using strategically located restriction sites and/or complementary sticky ends. The base vector for one approach according to embodiments of the present disclosure consists of plasmid with a multilinker in which all sites are unique (though this is not an absolute requirement). Sequentially, each promoter is inserted between its designated unique sites resulting in a base cassette with three promoters, or more, all of which can have variable orientation. Sequentially, again, annealed primer pairs are inserted into the unique sites downstream of each of the individual promoters, resulting in a triple expression cassette construct. The insert can be moved into, e.g. an AAV backbone using two unique enzyme sites (the same or different ones) that flank the triple expression cassette insert.

Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. The construct preferably comprises, for example, sequences necessary to package the miRNA expression construct into viral particles and/or sequences that allow integration of the promoter miRNA expression construct into the target cell genome. The viral construct also may contain genes that allow for replication and propagation of virus, though in preferred embodiments such genes will be supplied in trans. Additionally, the viral construct may contain genes or genetic sequences from the genome of any known organism incorporated in native form or modified. For example, the preferred viral construct comprises sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenicol acetyl transferase or secreted embryonic alkaline phosphatase; or proteins for which immunoassays are readily available such as hormones or cytokines. Other genetic elements that may find use in embodiments of the present disclosure include those coding for proteins which confer a selective growth advantage on cells such as adenosine deaminase, aminoglycodic phosphotransferase, dihydrofolate reductase, hygromycin-B-phosphotransferase, or those coding for proteins that provide a biosynthetic capability missing from an auxotroph. If a reporter gene is included along with the miRNA expression cassette, an internal ribosomal entry site (IRES) sequence can be included. Preferably, the additional genetic elements are operably linked with and controlled by an independent promoter/enhancer.

A viral delivery system based on any appropriate virus may be used to deliver the multiple-promoter miRNA expression constructs of the present disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to compounds as disclosed herein encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorus-containing and non-phosphorus-containing linkages are well known.

Peptide nucleic acids (PNAs) are short, artificially synthesized polymers with a structure that mimics DNA or RNA. PNAs include a backbone composed of repeating N-(2-aminoethyl)-glycine units linked by peptide bonds. Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences.

### Conjugates

In certain embodiments, provided herein are oligomeric compounds, which may, for example, comprise an miRNA oligonucleotide and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### Exosomes

Exosomes are small, relatively uniform-sized vesicles derived from cellular membranes. For example, exosomes may have a diameter of about 30 to about 150 nm. They contain several key proteins (e.g. CD9, CD63, CD81, CD82, Annexin, Flotillin, etc.) and in addition they package proteins, mRNAs, long non-coding RNAs, and miRNAs. Exosomes transport the payload from cell to cell. On entry into recipient cells the exosome payload is released into cytoplasm.

In some embodiments, at least one miRNA, or nucleic acid encoding one or more miRNAs, for treating, preventing, and/or controlling arthritis in a patient, as disclosed herein, is delivered to a cell via an exosome. Therefore, in one embodiment, an exosome carrying at least one miRNA, or nucleic acid encoding one or more miRNAs, for treating, preventing, and/or controlling arthritis in a patient as described herein is provided.

In one aspect, the invention is related to a pharmaceutical composition comprising a therapeutically effective amount of an exosome carrying at least one miRNA, or nucleic acid encoding one or more miRNAs, for treating, preventing, and/or controlling arthritis in a patient, as disclosed herein.

The exosome comprises an exosome-packaging-associated motif (also referred to as “exo-motif” hereinafter) operably linked, optionally through a linker, to the miRNA at least one miRNA, or nucleic acid encoding one or more miRNAs, for treating, preventing, and/or controlling arthritis in a patient, as disclosed herein. Another aspect of the invention is related to a pharmaceutical composition comprising a therapeutically effective amount of an exosome carrying at least one miRNA for treating, preventing, and/or controlling arthritis in a patient, and a pharmaceutically acceptable carrier, as disclosed herein. The exosome may comprise an exosome-packaging-associated motif operably linked, optionally through a linker, to the at least one miRNA.

As used herein, the term “microRNA”, “miRNA”, or “miR” refers to RNAs that function post-transcriptionally to regulate expression of genes, usually by binding to complementary sequences in the three prime (3′) untranslated regions (3′ UTRs) of target messenger RNA (mRNA) transcripts, usually resulting in gene silencing. miRNAs are typically small regulatory RNA molecules, for example, 21 or 22 nucleotides long. The terms “microRNA”, “miRNA”, and “miR” are used interchangeably.

The exosome of the present disclosure contains, for example, an effective amount of at least one miRNA, or nucleic acid encoding one or more miRNAs, treating, preventing, and/or controlling arthritis in a patient.

Another aspect of the disclosure provides a pharmaceutical composition comprising a therapeutically effective amount of an exosome as described above and a pharmaceutically acceptable carrier. In such aspect, a kit is provided to include the pharmaceutical composition in a single package. The kit may further include a specification for use that a physician can refer during clinical use.

Methods for transferring miRNAs, or nucleic acid encoding one or more miRNAs, into an exosome are available in the art, such as by co-transfecting a cell with a miRNA expression vector and a plasmid encoding miRNA. Other methods for packaging miRNAs into exosomes may also be applicable with the present disclosure.

In certain embodiments, the at least one miRNA, or nucleic acid encoding one or more miRNAs, or exosome comprising such nucleic acids made by a disclosed methods are administered by injection, use of scaffold or various matrices and glue forms, or by Carboplasty. In certain embodiments, the subject is human, racehorse or companion animal. The subject may be any animal.

In further embodiments, the injured site is a joint. In other embodiments, the at least one miRNA, or nucleic acid encoding one or more miRNAs, or exosome comprising such nucleic acids made by disclosed methods are administered to chronically damaged tissues in said subject for treating osteoarthritis and the pain associated with osteoarthritis. In some embodiments, the at least one miRNA, or nucleic acid encoding one or more miRNAs, or exosome comprising such nucleic acids made by a disclosed methods are administered by injection, by use of scaffold or various matrix and glue forms, or by Carboplasty. In certain embodiments, the subject is human, racehorse or companion animal.

The subject in need of at least one miRNA, or nucleic acid encoding one or more miRNAs, or exosome comprising such nucleic acids made by a disclosed methods treatment may be old, have low regenerative potential, or have low stem cell density, as frequently seen in female in comparison to male subjects; in need of cartilage regeneration in the joints to treat chondral (articular) cartilage damage, torn or damaged meniscus cartilage, torn or damaged labrum, subchondral bone edema, torn or damaged joint ligament, torn or damaged patellar cartilage, or an external injury to the joint.

This disclosure provides a method of tissue augmentation therapy comprising: administering a therapeutically effective amount of the at least one miRNA, or nucleic acid encoding one or more miRNAs, or exosome comprising such nucleic acids made by disclosed methods, to a subject in need thereof. In some embodiments, said at least one miRNA, or nucleic acid encoding one or more miRNAs, or exosome comprising such nucleic acids made by disclosed methods are administered to a non-injured site in said subject, wherein the subject in need of tissue augmentation or tissue reconstruction and the non-injured site comprise cartilage-containing tissues. The cartilage-containing tissues may be selected from any of the tip of the nose, ear and trachea.

In some embodiments, said at least one miRNA, or nucleic acid encoding one or more miRNAs, or exosome comprising such nucleic acids made by disclosed methods are administered by injection, by use of scaffold or various matrices, mesh and glue forms.

Any suitable route of administering the at least one miRNA, or nucleic acid encoding one or more miRNAs, or exosome comprising such nucleic acids made by the disclosed methods, may be used. In certain embodiments, the at least one miRNA, or nucleic acid encoding one or more miRNAs, or exosome comprising such nucleic acids made by the disclosed methods, or pharmaceutical composition comprising them, are administered by injection, scaffold or various matrix and glue forms, or by Carboplasty.

### Combination Therapy

The phrase “administering in combination” as used herein refers to any form of administration of one or more at least one miRNA, or nucleic acid encoding one or more miRNAs, or exosome comprising such nucleic acids made by disclosed methods and at least one additional therapeutic agent selected from the group consisting of tertiary or adjunctive agents within the formulations and methods of the invention include all known drugs and agents which are effective in relieving osteoarthritis and/or rheumatoid arthritis, and pain and inflammation caused by osteoarthritis and/or rheumatoid arthritis. Useful tertiary or adjunctive agents in this context include, but are not limited to, topical pain relievers including, but not limited to those containing methyl salicylate, menthol, camphor, eucalyptus and capsaicin; tramadol; acetaminophen; glucosamine; allopurinol; colchicine; demecolcine; oxypurinol; chondroitin; corticosteroid injections, including but not limited to glucocorticoids; and hyaluronic acid derivatives, including, but not limited to sodium hyaluronate and hylan G-F20. Adjunctive therapies may also be used including, but not limited to, physical treatments such as changes in diet, exercise, weight loss, heat treatment, cold treatment, acupuncture and surgery including, but not limited to, joint replacement, osteotomy, arthroscopic lavage and debridement, repositioning of bones, bone fusion, discectomy, and spinal fusion.

Useful NSAIDs within the formulations and methods of the disclosure include, but are not limited to, salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof.

### Pharmaceutical Compositions and Kits

An aspect of the disclosure provides a pharmaceutical composition comprising a therapeutically effective amount of oligonucleotide miRNA, an oligonucleotide encoding miRNA, an exosome comprising miRNA, a therapeutically effective amount of treating, preventing, and/or controlling arthritis in a patient and a pharmaceutically acceptable carrier. The pharmaceutical composition is useful for prophylaxis or treatment of arthritis in a subject. The pharmaceutical composition may be prepared in a suitable pharmaceutically acceptable carrier or excipient.

The present disclosure also provides pharmaceutical compositions comprising at least one miRNA, or nucleic acid encoding one or more miRNAs, or exosome comprising such nucleic acids made by methods as disclosed herein formulated together with one or more pharmaceutically or cosmetically acceptable excipients. These formulations include those suitable for oral, sublingual, intratracheal, intraarticular, intranasal, transdermal, pulmonary, intrathecal, intracisternal, parenteral (e.g., subcutaneous, intramuscular, intradermal, intraduodenal, or intravenous) or intralesional, administration, transmucosal (e.g., buccal, vaginal, and rectal), or for topical use, e.g., as part of a composition suitable for applying topically to skin and/or mucous membrane, for example, a composition in the form of a sterile aqueous solution, a sterile dispersion, a sterile powder, a lyophilized form, a gel, a paste, a wax, a cream, a spray, a liquid, a foam, a lotion, an ointment, an injectable solution, an injectable dispersion, a topical solution, a transdermal form, a transdermal patch, a powder, a vapor, a tincture, or combinations thereof. Although the most suitable form of administration in any given case will depend on the degree and severity of the condition being treated.

The present disclosure also provides a pharmaceutical composition comprising at least one miRNA, or nucleic acid encoding one or more miRNAs, or exosome comprising such nucleic acids made by disclosed methods, or a pharmaceutically acceptable salt thereof (for example, a miRNA that includes a nucleobase sequence of any of SEQ ID NOs: 1-38, or combinations thereof).

Under ordinary conditions of storage and use, these preparations/compositions may contain a preservative to prevent the growth of microorganisms. The pharmaceutical forms suitable for injectable use include sterile aqueous solutions or dispersions and sterile powders including the lyophilized form of at least one miRNA, or nucleic acid encoding one or more miRNAs, or exosome comprising such nucleic acids made by methods as disclosed herein for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, sodium chloride or phosphate buffered saline. Prolonged absorption of the injectable compositions can be brought about by the use in the compositions of agents delaying absorption, for example, aluminum mono stearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

The compositions as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Liquid formulations can also be prepared by dissolving or suspending one or the combination of active substances in a conventional liquid vehicle acceptable for administration so as to provide the desired dosage.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

### Dosage and Frequency of Administration

The dosage or amounts described below refer either to the at least one miRNA, or nucleic acid encoding one or more miRNAs, or exosome comprising such nucleic acids made by the disclosed methods or pharmaceutical compositions thereof.

In some embodiments, formulations include dosage forms that include at least 1 ng, at least 5 ng, at least 10 ng, at least 20 ng, at least 30 ng, at least 40 ng, at least 50 ng, at least 60 ng, at least 70 ng, at least 80 ng, at least 90 ng, at least 100 ng, 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of, for example, a miRNA oligonucleotide. In some embodiments, formulations include dosage forms that include from 10 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a STMN2 antisense oligonucleotide.

In some embodiments, formulations include dosage forms that include or consist essentially of about 10 mg to about 500 mg of miRNA. For example, formulations that include about 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed miRNA. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed miRNA. The amount administered will depend on variables such as the type and extent of disease or indication to be treated, the overall health and size of the patient, the in vivo potency of the miRNA, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once every 4 weeks, once every 5 weeks, once every 6 weeks, once every 7 weeks, once every 8 weeks, once every 9 weeks, once every 10 weeks, once every 11 weeks, or once every 12 weeks. In some embodiments, dosing is once a month to every three months.

The invention will be illustrated in more detail with reference to the following Examples, but it should be understood that the present invention is not deemed to be limited thereto.

### EXAMPLES

### Example 1—Method of Extraction of Synovial Fluid

The procedure is performed in sterile conditions, typically using a 27-gauge needle and a 20-ml syringe or the alternative depending on the surgeon's specifications. To mobilize the free floating synovial fluid cells, the joint is gently flexed and extended before the harvest. The aspirate is often an amber color solution, but at times it can include small amounts of blood. The cell count and cytometry analysis discount the red and white blood cells at the time of culture. Using this procedure, on average, 0.5-5M cells with 85-98% viability are obtained from each harvest. The harvesting of synovial fluid can also be done concurrent with portal arthroscopy, should the subject need to have this procedure done for other reasons.

### Example 2—Method of Expansion of Synovial Fluid-Derived Stem Cells

Synovial fluid is obtained from subjects and processed under sterile conditions in a laminar flow hood. Each sample is centrifuged at 1500 rpm for 10 minutes to obtain a pellet. The cell pellets (including all cells harvested) are suspended in DMEM (Dulbecco's Minimum Essential Medium—GIBCO), supplemented with 20% fetal bovine serum and 1% antibiotic-antimycotic (Gibco-Life Technologies). Cells are counted and cultured in T-75 flasks and incubated at 37° C. and 5% CO2 for 48 hours. The cultures are then fed every 48 hours until they reached 90% confluency in 2-3 weeks. At the time of the harvest, synovial fluid mesenchyme stem cells positive for CD73, CD90 and CD105 markers (SF-MSCs), comprise 0.5-3% of the total population present in synovial fluid. At the end of the PO culture (at confluence in 2-3 weeks), this number increases to about 80-94%. SF-MSCs form colonies and take over the culture.

### Example 3—Method of Harvesting Exosomes

Exosomes are isolated from culture media of synovial fluid-derived mesenchyme stem cell cultures (at 2nd or 3rd passage) when confluence has been reached. 48 hour prior to harvesting of exosomes, cells are treated with serum-free media to exclude presence of any serum-derived exosomes. Exosomes are isolated from the culture media by using ultrafiltration devices (Vivaspin 20) containing 100 kDa molecular weight cut-off (MWCO) PES membrane and spinning at 3,000×g for 5 min. Any remaining retentate, as well as the eluate, are discarded. The sample is then loaded into pre-rinsed centrifugal filter devices, 15 mL per device, and centrifuged at 3000×g for several intervals of 30 minutes until the final volume reaches 500 μL.

Size Exclusion Separation is used to resolve particles by size. Larger particles eluded first on the column, followed by proteins and small molecular weight compounds. The exosome fractions are concentrated using Amicon Ultra 2 100 kDa MWCO centrifugal filter devices. A total of 180 μL was recovered after centrifugation and transferred into a new tube. The extracted exosomes were kept in −80° C. until used.

### Example 4—Method of Exosomes Tracking

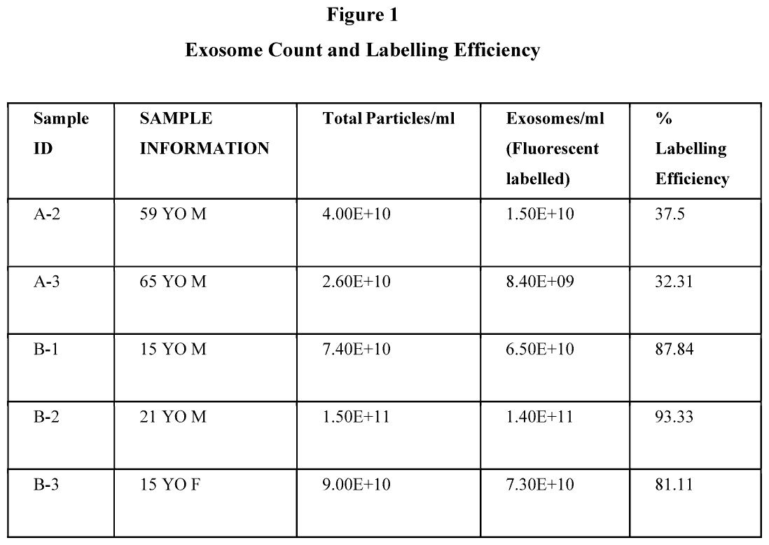

Fluorescent Nanoparticle Tracking Analysis (fNTA) technique was used to count and measure size distribution of intact exosomes. This technique involves labeling of exosome membranes using cell mask deep red (CMDR), and performing the analysis in scatter and fluorescent modes. The fNTA technique allows exclusion of contaminant particles, such as protein aggregates, lipoproteins, etc. from analysis and assesses purity of exosome samples. Our analysis was performed using Zetaview Quatt (Particle Metrix) instrument. DI water and PBS were filtered on the day of analysis through 0.22 μm syringe filter and their purity confirmed by NTA prior to the study. CMDR working solution of 0.05 mg/mL was prepared by adding 1 μL of 5 mg/mL stock to 100 μL freshly filtered PBS. 1 μL of the working solution was added to 19 μL of sample. Dilutions were made by mixing PBS filtered through 0.2 μm syringe filter with corresponding volume of a sample. (See FIG. 1).

The following instrument settings were used:

| | Scatter | Fluorescent |
|---|---|---|
| Mode | (520 nm) | (640/660 nm) |
| Sensitivity | 80 | 89 |
| Shutter | 100 | 100 |
| Cycles/positions | 2/11 | 1/11 |
| Frame rate | 30 | 30 |
| Maximum Size | 1000 | 1000 |
| Minimum area | 10 | 10 |
| Track Length | 15 | 7 |
| Minimum Brightness | 20 | 15 |

### Example 5—Method of Exosomal RNA Isolation

RNA molecules were isolated using Exosomal RNA isolation Kit (NorgenBiotek) according to manufacturer's protocol. RNA concentration and Quality Control were done using BioAnalyzer. After RNA isolation and QC, larger RNAs (more than 100 nucleotides) were filtered out and 40-50 nucleotide long RNAs (miRNA, some tRNA, etc.) were mapped to different biotypes by Next Generation Sequencing.

### Example 6—Method of RNA Next Generation Sequencing (NGS) Run and Mapping

The Small RNA Library Prep Kit for Illumina was used for RNA mapping. NextSeq 500/550 High Output Kit v2 (75-Cycle Kit) was used as the sequencing platform. Library preparation workflow included 3′ and 5′ adapter ligation, followed by reverse transcription and the indexing PCR. Library QC was performed using Agilent Bioanalyzer pico chip to estimate library size and concentration. Libraries were then pooled, denatured and diluted to required concentration. Libraries were then applied onto a flowcell and sequenced using Illumina platform. Initial data Analysis included adapter trimming, QC and filtering. The reads were then mapped to the whole genome. Read counts for small RNAs were normalized using Counts Per Million as normalization method where the read of a specific small RNA species were divided by the total number of mapped reads and multiplied by one million. For differential Expression analysis trimmed mean of M-values (TMM) normalization method was used. TMM normalization takes into account variations in sequencing depth and library size. It also uses scale factors between samples to correct counts and overcome under-sampling effects caused by highly expressed miRNAs.

### Example 7—Method of Differential Expression of miRNA

Patient Samples from different age groups were subjected to differential miRNA expression analyses. Two subjects were placed in the A (old) group: 59 yo Male (code name A2) and a 65 yo male (code name A3). Three subjects were placed in the B (young) group: 15 yo male (code name B1); 21 yo male (code name B2) and 15 yo female (code name B3). Groups A and B were referred to “old” and “young”, respectively, for age variability analysis purposes.

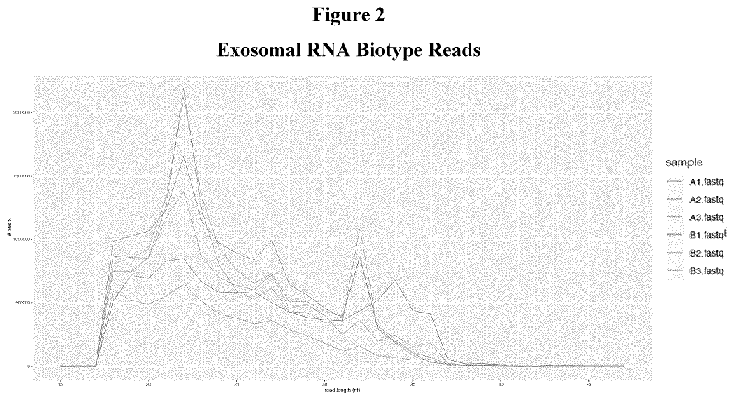

Read length distribution based on normalized raw reads indicated that the most frequent occurrence for all groups was at 22 nucleotides (miRNA) peak. Second highest peak occurred at 32 nucleotides (tRNA). Both peaks showed higher number of reads in group B (young) than group A (old), indicating higher miRNA and tRNA counts/million in Group B. (See FIG. 2)

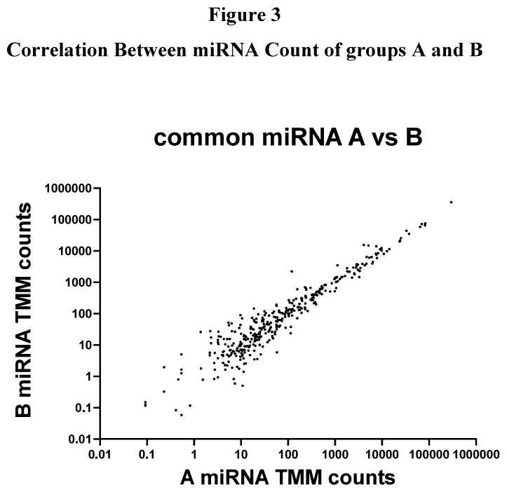

For differential Expression analysis trimmed mean of M-values (TMM) normalization method was used to overcome under-sampling effects and reduce false-positive rate. Using TMM method, the variabilities among the miRNAs of group “young”, B vs group “old”, A were assessed. The study demonstrated that miRNAs with lowest count showed higher variability of expression among groups than those that occur at higher count (See FIG. 3)

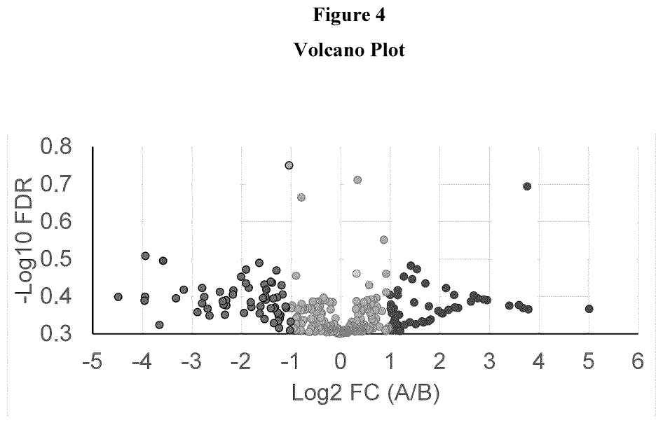

Volcano plot of TMM normalized FDR (the Fold Discovery Rate) or adjusted p-Value (the Benjamini-Hochberg procedure) was used to show variability of miRNA expression between groups A and B. Of the 300 or so miRNAs observed in groups A and B, the middle area, represented less that 2× difference of occurrence between groups. Markers on the right, represented higher occurrence in group A than B (A/B>1). Markers on the left represent higher variability of miRNA expression in Group B than A (A/B<1). (See FIG. 4).

### Example 8—Venn Diagram

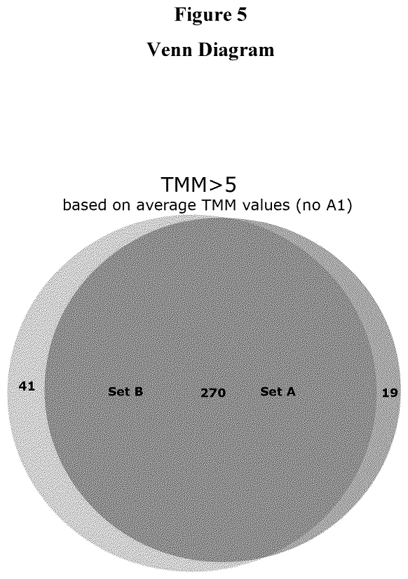

The Venn Diagram, showed the logical relationships between min 5 TMM normalized counts miRNAs of all groups. Of the total miRNAs, 270 were commonly expressed in both groups A, “old” and B, “young”. On the other hand, 41 miRNAs were unique to group B, “young” and 19 miRNAs were unique to group—A “old”. (See FIG. 5).

### Example 9—Bioinformatics Analysis of miRNA Unique to Group B

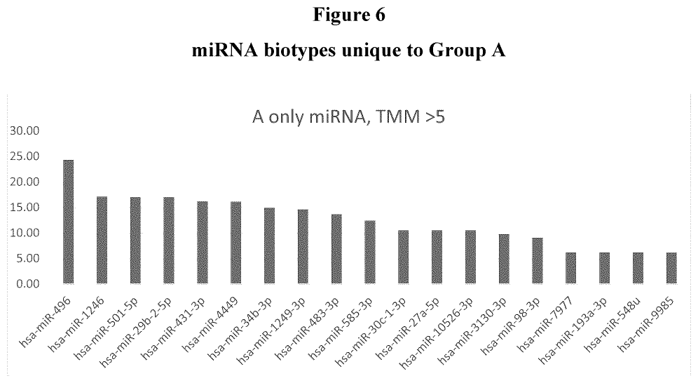

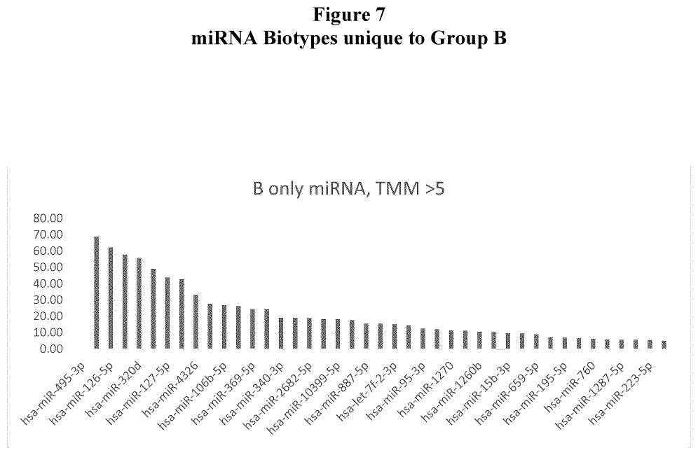

Bioinformatics analysis was performed to rank the miRNAs unique to groups A, “old” and group B, “young” in order of highest to lowest concentration. (See FIGS. 6 and 7 respectively)

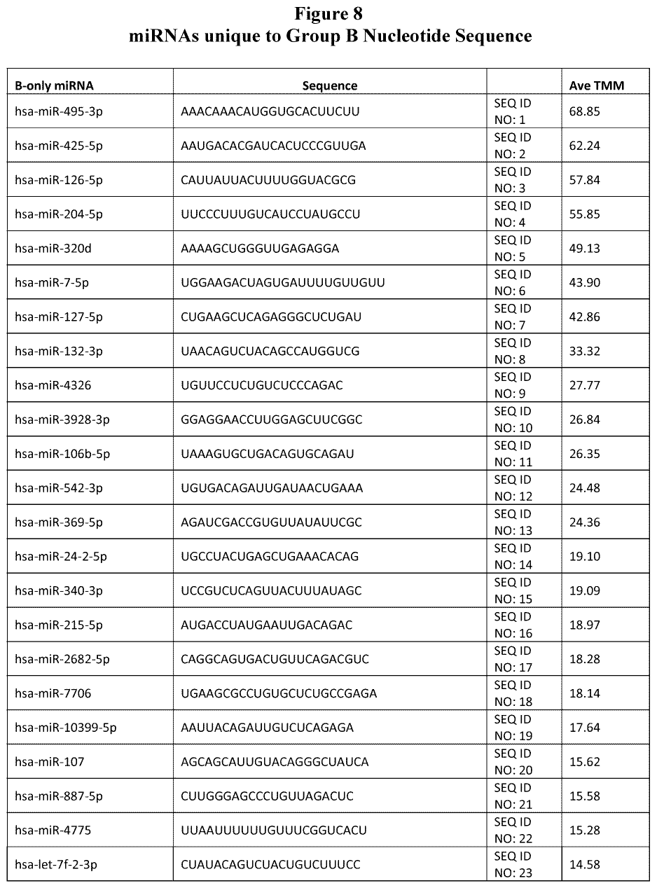

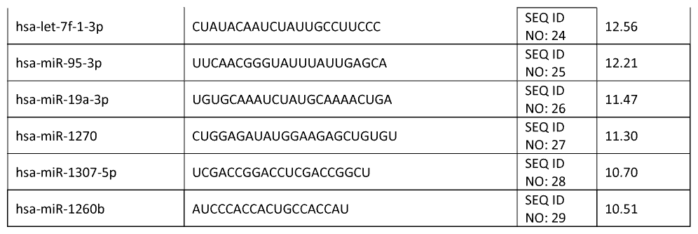

We then determined the nucleotide sequence of the miRNAs unique to group B, “young” by reference to the miRBase: the microRNA database (www.mirbase.org). (See FIG. 8).

### Example 10—Bioinformatics Analysis of tRNAs of Groups A and B

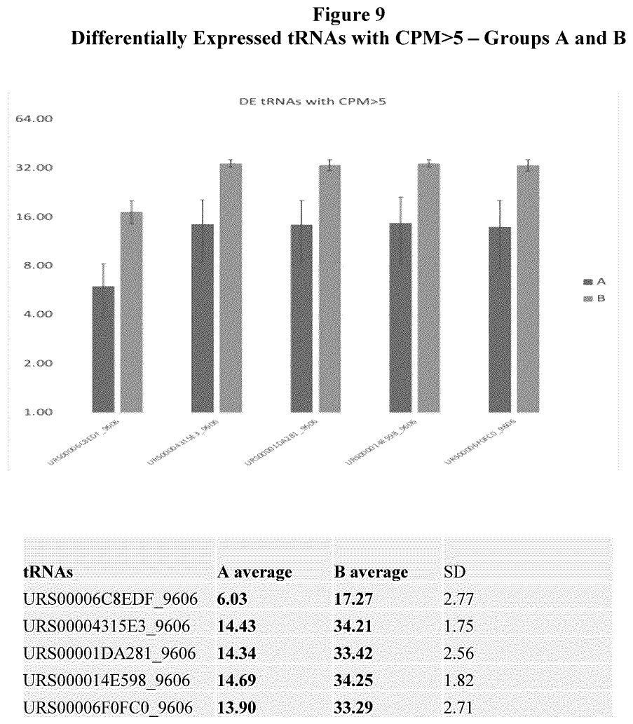

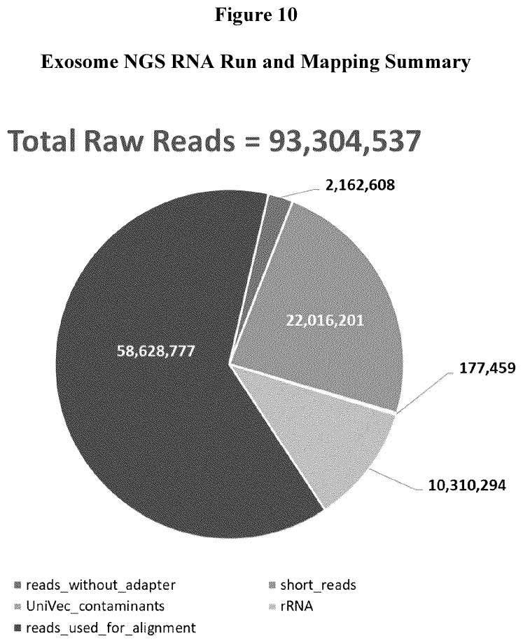

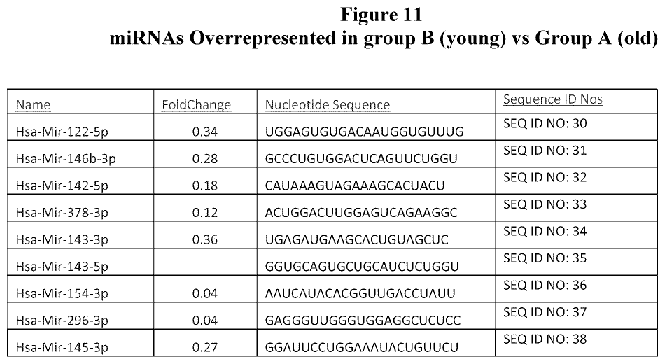

Bioinformatics analysis was performed to determine the min 5 CPM normalized count tRNAs of groups A and B. The top 5 highly expressed tRNAs were assessed. In all, tRNA counts for group B (young) were higher than those of group A (old). (See FIG. 9).

### Example 11—Method of Implantation of Exosomes and/or Associated Signaling Factors

Extracted exosomes and/or their associated signaling factors including miRNAs, tRNAs and other molecules found in the selected miRNAs will be administered by direct injection in synovial cavity, embedded in scaffoldings (for example Hyalofast Scaffolding) or various matrix and glue forms, or by Carboplasty. The treated sites will be checked for outcomes on months 1.5, 3, 6 and 12 post-implantation. Mini Koos will be used as the pain scale and Pre- and post MRI scanning for edema reduction and CartiGram (T2 mapping sequence) to map increase in cartilage thickness. (See for example U.S. 2021/0268031 at FIG. 4A-E)

### Pharmaceutical Compositions and Formulations:

Compositions containing exosomes or miRNAs or tRNAs made by the disclosed methods can be formulated as a pharmaceutical composition for administering to a subject. Any suitable pharmaceutical compositions and formulations, as well as suitable methods for formulating and suitable routes and suitable sites of administration, are within the scope of this invention, and are known in the art. Also, unless otherwise stated, any suitable dosage(s) and frequency of administration are contemplated.

The pharmaceutical compositions can include a pharmaceutically acceptable carrier (i.e., an excipient). A “pharmaceutically acceptable carrier” refers to, and includes, any and all solvents, dispersion media, coatings, antibacterial and antifungal agents, isotonic and absorption delaying agents, diluent, glidant, etc. The compositions can include a pharmaceutically acceptable salt, e.g., an acid addition salt or a base addition salt (see e.g., Berge et al. (1977) J Pharm Sci 66:1-19). The composition can be coated when appropriate.

Any suitable dosage may be used.

Any suitable route of administering the exosomes or their associated miRNAs or tRNAs made by the disclosed method may be used. In certain embodiments, the exosomes made by the disclosed method, or pharmaceutical composition comprising either, are administered by injection, scaffold or various matrix and glue forms, or by Carboplasty.

While the invention has been described in detail and with reference to specific examples thereof, it will be apparent to one skilled in the art that various changes and modifications can be made therein without departing from the spirit and scope thereof.

What is claimed is:

1. A method of treating, preventing, and/or controlling arthritis and the pain associated with arthritis in a patient, the method comprising:
selecting a patient in need of treating, preventing, and/or controlling arthritis, torn or damaged meniscus cartilage, torn or damaged labrum, subchondral bone edema, torn or damaged joint ligament, torn or damaged patellar cartilage, or an external injury to the joint;
administering to the patient a therapeutically effective amount of a pharmaceutical composition comprising at least one of the following:
(i) at least one miRNA selected from the group consisting of SEQ ID NO: 1 to SEQ ID NO: 38, and combinations thereof;
(ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect;
(iii) at least one miRNA having 80% or more sequence identity to a miRNA selected from the group consisting of SEQ ID NO: 1 to SEQ ID NO: 38, and combinations thereof, and having a therapeutic effect;
(iv) a nucleic acid encoding at least one miRNA selected from the group consisting of SEQ ID NO: 1 to SEQ ID NO: 38, and combinations thereof;
(v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect;
(vi) a nucleic acid encoding at least one miRNA having 80% or more sequence identity to a miRNA selected from the group consisting of SEQ ID NO: 1 to SEQ ID NO: 38, and combinations thereof, and having a therapeutic effect, or
(vii) combinations thereof.

2. The method according to claim 1, wherein said arthritis is osteoarthritis or rheumatoid arthritis.

3. The method according to claim 1 wherein the subject in need of treatment has low chondrogenic potential or has small stem cell population as in the gender-specific and age-specific stem cell count variabilities.

4. The method according to claim 1 wherein the subject is in need of tissue reconstruction or tissue augmentation therapy, including the treatment of acute and chronic wounds that have afflicted layers of connective and epidermal tissue of the body, or for aesthetic reasons, including reduction of wrinkles, grooves, scars, acne scars, traumatic scars, sequelae of cellulite, as well as for other irregularities of the skin, to give a smoother skin.

5. The method according to claim 1 wherein the patient in need of treating, preventing, and/or controlling arthritis and the pain associated with arthritis is selected from the group consisting of a human, a horse, or a companion animal.

6. The method according to claim 1 wherein said miRNA is chemically modified.

7. The method according to claim 6 wherein said chemical modification is one or more chemical modifications selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof.

8. The method according to claim 1 wherein the miRNA comprises a modified backbone.

9. The method according to claim 1 wherein the miRNA comprises a phosphorothioate backbone or a phosphorodithioate backbone.

10. The method according to claim 1 wherein the pharmaceutical composition further comprises a nucleic acid transfection agent.

11. The method according to claim 10 wherein said transfection agent is a lipid-based transfection agent, a polymer-based transfection agent, a magnetic particle-based transfection agent, an exosome for nucleic acid delivery, or a viral protein for nucleic acid delivery.

12. The method according to claim 1 wherein the pharmaceutical composition is for topical administration.

13. The method according to claim 1 wherein the pharmaceutical composition is formulated for parenteral administration.

14. The method according to claim 1 wherein the pharmaceutical composition is formulated for intra-articular, intravenous, intramuscular, sub-cutaneous, or intraperitoneal injection.

15. The method of according to claim 1 wherein said pharmaceutical composition is administered by a mode selected from the group consisting of injection, a scaffold, a 3-D scaffold, a matrix and a glue, carboplasty, and combinations thereof.

16. The method according to claim 1 further comprising co-administration with other antiarthritis agents.

17. The method according to claim 15 wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, and combinations thereof.

18. A pharmaceutical composition for treating, preventing, and/or controlling arthritis in a patient comprising at least one of the following:
(i) at least one miRNA selected from the group consisting of SEQ ID NO: 1 to SEQ ID NO: 38, and combinations thereof;
(ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect;
(iii) at least one miRNA having 80% or more sequence identity to a miRNA selected from the group consisting of SEQ ID NO: 1 to SEQ ID NO: 38, and combinations thereof, and having a therapeutic effect;
(iv) a nucleic acid encoding at least one miRNA selected from the group consisting of SEQ ID NO: 1 to SEQ ID NO: 38, and combinations thereof;
(v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect;
(vi) a nucleic acid encoding at least one miRNA having 80% or more sequence identity to a miRNA selected from the group consisting of SEQ ID NO: 1 to SEQ ID NO: 38, and combinations thereof, and having a therapeutic effect, or
(vii) combinations thereof.

19. The pharmaceutical composition according to claim 18, wherein said arthritis is osteoarthritis or rheumatoid arthritis.

20. The pharmaceutical composition according to claim 18 wherein said miRNA is a pri-miRNA, a pre-miRNA, a double-stranded miRNA, a single-strand miRNA expressed from the 5′-end of a pre-miRNA, or a single-strand miRNA expressed from the 3′-end of a pre-miRNA.

21. The pharmaceutical composition according to claim 18 wherein said miRNA is chemically modified.

22. The pharmaceutical composition according to claim 21 wherein said chemical modification is selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof.

23. The pharmaceutical composition according to claim 18 wherein the miRNA comprises a modified backbone.

24. The pharmaceutical composition according to claim 18 wherein the miRNA comprises a phosphorothioate backbone or a phosphorodithioate backbone.

25. The pharmaceutical composition according to claim 18 wherein the pharmaceutical composition further comprises a nucleic acid transfection agent.

26. The pharmaceutical composition according to claim 25 wherein said transfection agent is a lipid-based transfection agent, a polymer-based transfection agent, a magnetic particle-based transfection agent, an exosome for nucleic acid delivery, or a viral protein for nucleic acid delivery.

27. The pharmaceutical composition according to claim 18 wherein the composition is for topical administration.

28. The pharmaceutical composition according to claim 18 wherein the composition is formulated for parenteral administration.

29. The pharmaceutical composition according to claim 18 wherein the composition is formulated for intra-articular, intravenous, intramuscular, sub-cutaneous, or intraperitoneal injection.

30. The pharmaceutical composition according to claim 18 wherein the composition is co-administered in combination with other antiarthritis agents.

31. The pharmaceutical composition according to claim 30 wherein said other antiarthritis agents are one or more antiarthritis agents selected from the group consisting of wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifunmidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof.

32. A pharmaceutical composition comprising an exosome and an excipient, wherein the exosome comprises:
(i) at least one miRNA selected from the group consisting of SEQ ID NO: 1 to SEQ ID NO: 38, and combinations thereof;
(ii) at least one miRNA having a substitution, addition, and/or deletion of 1-5 bases to a miRNA selected from the group consisting of SEQ ID NO: 1 to SEQ ID NO: 38, and combinations thereof, and having a therapeutic effect;
(iii) at least one miRNA having 80% or more sequence identity to a miRNA selected from the group consisting of SEQ ID NO: 1 to SEQ ID NO: 38, and combinations thereof, and having a therapeutic effect;
(iv) a nucleic acid encoding at least one miRNA selected from the group consisting of SEQ ID NO: 1 to SEQ ID NO: 38, and combinations thereof;
(v) a nucleic acid encoding at least one miRNA having a substitution, addition, and/or deletion of 1-5 bases to a miRNA selected from the group consisting of SEQ ID NO: 1 to SEQ ID NO: 38, and combinations thereof, and having a therapeutic effect; or
(vi) a nucleic acid encoding at least one miRNA having 80% or more sequence identity to a miRNA selected from the group consisting of SEQ ID NO: 1 to SEQ ID NO: 38, and combinations thereof, and having a therapeutic effect.

33. The pharmaceutical composition of claim 32, wherein the composition is formulated for parenteral administration.

34. The pharmaceutical composition according to claim 32 wherein the composition is formulated for intra-articular, intravenous, intramuscular, sub-cutaneous, or intraperitoneal injection.

35. The pharmaceutical composition according to claim 32 wherein the miRNA comprises a modified backbone.

36. The pharmaceutical composition according to claim 32 wherein the miRNA comprises a phosphorothioate backbone or a phosphorodithioate backbone.

37. The pharmaceutical composition according to claim 32 wherein the miRNA comprises one or more chemical modifications selected from the group consisting of LNA-tion, BNA-tion, ENA-ation, 2′-OMe modification, a 2′-O-methyl ribonucleotide, a 2′-deoxy-2′-fluoro ribonucleotide, a “universal base” nucleotide, a 5-C-methyl nucleotide, a phosphorothioate internucleotide linkage, and an inverted deoxyabasic residue incorporation, phosphorothioation, S-TuD-ation, morpholino modification, peptide addition, glycosylation, aptamer addition, hydrophobic molecule addition, polymer addition, addition of unmodified DNA, and combinations thereof.

38. The pharmaceutical composition according to claim 32 wherein the composition is used in combination with other antiarthritis agents.

39. The pharmaceutical composition according to claim 38 wherein said other antiarthritis agents are one or more antiarthritis agents selected from the group consisting of wherein said other antiarthritis agents are one or more agents selected from the group consisting of salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifunmidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof.

40. The pharmaceutical composition of claim 32 wherein the pharmaceutical composition is in a form selected from the group consisting of a sterile aqueous solution, a sterile dispersion, a sterile powder, a lyophilized form, a gel, a paste, a wax, a cream, a spray, a liquid, a foam, a lotion, an ointment, an injectable solution, an injectable dispersion, a topical solution, a transdermal form, a transdermal patch, a powder, a vapor, a tincture, and combinations thereof.

41. A method of identification of signaling factors/biologic factors which can be used to induce cartilage growth in a patient in need thereof, the method comprising:
Providing an older patient;
isolating synovial fluid from the older patient;
Isolating signaling factors from the synovial fluid, or cells or cell derivatives of synovial fluid, adipose tissue or bone marrow of adults, cells from embryo, fetuses, placentas, umbilical cord, Wharton Jelly or a mixture thereof, of the older patient;
providing a younger patient;
isolating synovial fluid from the younger patient,
isolating signaling factors from the synovial fluid, or cells or cell derivatives of synovial fluid, adipose tissue or bone marrow of adults, cells from embryo, fetuses, placentas, umbilical cord, Wharton Jelly or a mixture thereof, of the younger patient;
performing a comparison of the signaling factors isolated from the synovial fluid, or cells or cell derivatives of synovial fluid, adipose tissue or bone marrow of adults, cells from embryo, fetuses, placentas, umbilical cord, Wharton Jelly or a mixture thereof of the younger patient to the signaling factors isolated from the synovial fluid, or cells or cell derivatives of synovial fluid, adipose tissue or bone marrow of adults, cells from embryo, fetuses, placentas, umbilical cord, Wharton Jelly or a mixture thereof from the older patient;
identifying the signaling factors which are unique to the synovial fluid, or cells or cell derivatives of synovial fluid, adipose tissue or bone marrow of adults, cells from embryo, fetuses, placentas, umbilical cord, Wharton Jelly or a mixture thereof of the younger patient;
wherein the unique signaling factors from the younger patient are useful for growing cartilage.

42. The method of claim 41 wherein the younger patient is under about 25 years of age.

43. The method of claim 41 wherein the older patient is over about 55 years of age.

44. The method of claim 32 wherein the signaling factors are selected from the group consisting of Synovial Fluid-Derived Mesenchyme Stem Cells, Exosomes, Exosomal RNA, Exosomal miRNA, Exosomal tRNA, Exosomal peptides and combinations thereof.
